(19) United States
(12) Patent Application Publication
(10) Pub. No.: US 2013/0110048 A1
(43) Pub. Date: May 2, 2013

(54) PATIENT SITE PROTECTIVE COVER

(71) Applicant: MTM INNOVATIVE SOLUTIONS, LLC, Florence, KY (US)

(72) Inventor: Anthony Herzog, Erlanger, KY (US)

(73) Assignee: MTM INNOVATIVE SOLUTIONS, LLC, Florence, KY (US)

(21) Appl. No.: 13/658,225

(22) Filed: Oct. 23, 2012

Related U.S. Application Data
(60) Provisional application No. 61/554,204, filed on Nov. 1, 2011.

(51) Int. Cl. A61M 25/02

(52) U.S. Cl. 604/179

(57) ABSTRACT

A patient site protective cover comprising a membrane, a resilient and substantially impermeable seal around a perimeter of the membrane and dimensioned to surround a site of a patient. At least one strap is operably coupled to the membrane and configured to secure the cover over the site.

### CROSS-REFERENCE TO RELATED APPLICATIONS

This application claims the benefit of and priority to prior filed co-pending U.S. Provisional Patent Application Ser. No. 61/554,204 filed Nov. 11, 2011, which is expressly incorporated herein by reference.

### FIELD OF THE INVENTION

This invention relates to methods and apparatus for protecting sites on a patient from contamination.

### BACKGROUND OF THE INVENTION

Traditionally, devices that protect intravenous (IV) ports or IV tubing have not provided coverage or waterproofing sufficient to allow patients to bathe or shower without withdrawing and re-inserting the intravenous needle. Repeating the withdrawal and reinsertion process prior to every encounter with moisture or other contaminants can be time-consuming, painful, and expensive. For example, conventional devices claim to waterproof the site by utilizing an adhesive patch. These devices are non-reusable and are difficult to remove without causing discomfort or trauma to the injection site. The patch type products are also unable to adequately protect the entire IV tubing system while the patient is in a contaminating environment. Alternatively, other systems use a waterproof sleeve that envelops the entire arm. These full sleeve-covers limit the patient's range of motion and tend to be uncomfortable.

Therefore, there exists a need for a device that will protect IV ports and tubing during exposure to contaminating environments, and that is reusable, inexpensive, waterproof, and does not significantly interfere with the patient's activities.

### SUMMARY OF THE INVENTION

A patient site protective cover comprises a membrane, a resilient and substantially impermeable seal around a perimeter of the membrane and dimensioned to surround a site of a patient, and a stiffening member positioned around at least a portion of the perimeter of the membrane. The stiffening member is configured to resist dimpling and distortion of the membrane. The cover also includes at least one strap operably coupled to the membrane and configured to secure the cover over the site.

The device might include a membrane that is substantially impermeable to liquid, vapor, and particulate matter. Alternatively, the cover may include a membrane that is differentially permeable and configured to allow vapor to pass through the membrane, and further configured to prevent liquids or particulate matter from passing through the membrane. Further still, the cover could include a membrane that is configured to allow vapor and liquid to pass through the membrane, and further configured to prevent particulate matter from passing through the membrane.

The cover may include a stiffening member that is resilient and has memory, thereby allowing a user to substantially conform the cover to contours of the site. Additionally, the cover might include at least one buckle operably coupled to at least one strap and configured to tension the strap. The strap may include an elastic portion or may use a hook and loop fastener system.

The device may utilize a seal comprised of a plurality of protrusions or bulbs configured to improve the interface between the seal and the site. Additionally, the cover could be coated with an adhesive or a lubricant to improve the interface between the seal and the site. Further, the seal can be dimensioned to produce a void between the membrane and the site, or dimensioned with a height effective to support the membrane in a non-contact relationship relative to the higher of one of the site, and an object penetrating the site.

A method of protecting a site on a patient from contamination is provided. The method includes the steps of surrounding the site with a substantially impermeable seal, placing a membrane over the site and the seal, utilizing a stiffening member positioned around at least a portion of a perimeter of the membrane, where the stiffening member is configured to resist dimpling and distortion of the membrane. The method further includes the steps of bonding the seal, membrane, and stiffening member together, and securing the seal, membrane, and stiffening member to the site with at least one strap.

The method could include placing the membrane maintaining a non-contacting relationship between the membrane and the site or the step of dimensioning the seal to provide a void between the membrane and the higher of one of the site, and an object penetrating the site. The method may also include the step of storing a length of tubing, connected to an IV/catheter penetrating the site, within the void. Likewise, the step of placing the membrane could include selecting a membrane that is substantially impermeable to liquid, vapor, and particulate matter. Further, the step of placing the membrane might include selecting a differentially permeable membrane that is configured to allow vapor to pass through the membrane, and further configured to prevent liquids or particulate matter from passing through the membrane.

A patient site protective cover comprising a membrane, a resilient and substantially impermeable seal around a perimeter of the membrane and dimensioned to surround a site of a patient, and a stiffening member positioned around at least a portion of the perimeter of the membrane, is provided. The stiffening member is configured to resist dimpling or distortion of the membrane. The cover further includes a pair of mounting tabs, wherein each mounting tab is positioned at a respective opposite side edge of the membrane and each mounting tab has at least one slot in each opposite ends of itself. The cover also includes a plurality of straps configured to secure the cover over the site, where each strap of the plurality of straps is threaded through the slots in the respective ends of the mounting tabs.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a bottom plan view of the Patient Site Protective Cover.

FIG. 2 is a top plan view of the device of FIG. 1.

FIG. 3 is a cross sectional view, taken along line 3-3 of FIG. 1.

FIG. 4 is a front of the device in use.

FIG. 5 is a rear view of the device in use.

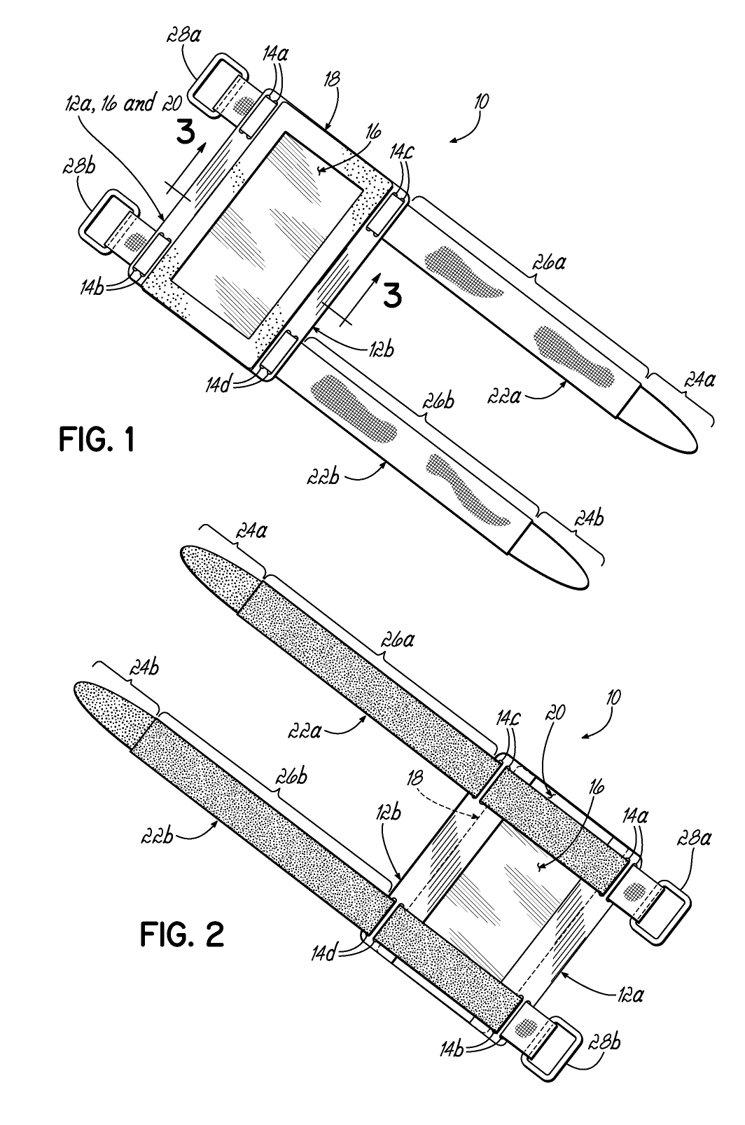

The components labeled in FIGS. 1-5 are outlined below and described in detail in the Detailed Description Section

### DETAILED DESCRIPTION

This particular invention relates to a Patient Site Protective Cover (hereafter “cover”) that has special benefits for the ambulatory patient while subjected to a contaminating environment. For patients undergoing intravenous infusion therapy, this cover is intended to stabilize and protect the catheter/IV and associated tubing from water, moisture and other contaminates. The cover will allow patients to bathe or shower without withdrawing and re-inserting the intravenous needle, which can be time-consuming, painful and expensive.

The cover may be manufactured in variety of sizes to physically stabilize and secure the catheter IV tubing system for any size patient. While the cover is specifically illustrated herein as being used to protect an infusion site on the arm, it would be readily appreciated that the cover may be adapted to protect any site on the patient from contamination. Accordingly, the cover may be readily adapted to protect and reduce contamination of wounds, burns, or objects impaled through the skin, regardless of location. Further, the cover is equally suited to site protection on human and veterinary patients. Therefore, throughout the discussion below, it should be observed that apparatus and methods for protecting a site on a patient, are expressly defined to include veterinary applications.

Referring to FIGS. 1-3, a cover 10 includes mounting tabs 12a and 12b, which serve as a foundation for mounting additional disclosed elements. The mounting tabs 12a and 12b are constructed of a rigid or semi-rigid material (phenyl ethane, polyvinyl chloride, aluminum, or the like), and are configured to mate adjustable attachment components to the waterproof sealing components (explained in detail below). In one embodiment, the mounting tabs 12a and 12b include slots 14a-14d to facilitate mounting of adjustable attachment components.

A membrane 16 is coupled to the mounting tabs 12a and 12b, wherein the mounting tabs 12a and 12b are generally affixed at opposing edges of the membrane 16. The membrane 16 is configured to act as a barrier to protect structures situated between the patient and the cover 10, and it may be fabricated from a substantially impermeable material, for example, polyvinyl chloride, latex, or butyl rubber. While substantially impermeable materials possess effective waterproofing characteristics, prolonged use may result in retention of perspiration between a patient's skin and the membrane 16 of the cover 10. Therefore, certain applications may benefit from materials that allow perspiration or solvent vapor to pass from the patient to the environment, but prevent liquids from traveling from the environment to the patient. Therefore, in another embodiment, the membrane 16 may comprise a differentially permeable membrane (e.g., Gortex® or Conduit®). In yet another embodiment, the membrane 16 may be comprised of paper or cloth of sufficient porosity to protect against contamination by particulate matter. The membrane 16 may utilize transparent materials (to facilitate visual inspection of the site), translucent materials, or opaque materials and may be flexible or substantially rigid. If a substantially rigid material is selected (one that is not prone to distortion under normal operating conditions), the membrane 16 may be formed with a curved geometry to accommodate placement on an appendage. Likewise, a rigid membrane 16 may include a dome or other non-planar feature to provide a void or clearance between a portion of the membrane 16 and the site it is affixed to. The various materials selected for the membrane 16 are illustrative only, and other suitable protective materials may be implemented.

Coupled to the membrane 16 is a seal 18. In one embodiment, the seal 18 is formed from a seamless ring of resilient material. To provide a protective seal between the skin of the patient and the membrane 16, the seal 18 may be fashioned from a variety of hydrophobic and substantially impermeable materials, to include neoprene, EDPM, silicone, closed wall foam, or polyvinyl chloride. The shape of the seal 18 may vary from an elliptical, rectangular, or irregular geometry to accommodate any therapeutic device that may penetrate the skin. For example, orthopedic screw-heads, wound drain tubes, or other impalements of the skin may be effectively protected from contamination with an appropriately dimensioned seal 18. In some embodiments, the height of the seal 18 is configured to maintain a non-contact relationship between the membrane 16 and the site or skin of the patient. In other embodiments, additional seal height is added to maintain a non-contact relationship between the membrane 16 and an object penetrating the site. The cross sectional profile of the seal 18 may include rectangular, circular, elliptical, square, single protrusion (bulb), or a plurality of protrusions. By way of example, a seal 18 with multiple protrusions or bulbs may have a cross sectional area similar to a capital letter “B” or “W”. Multiple protrusions afford the seal 18 an opportunity to contact the site at in a plurality of locations along a given cross sectional region, and may therefore improve sealing efficacy. The seal 18 may be coated with a substance such as petroleum jelly, coconut butter, Shea butter, adhesive, etc., to improve waterproofing characteristics and to enhance the interface between the seal 18 and the patient's skin.

In one embodiment, the perimeter region of the membrane 16 includes a stiffening member 20. The stiffening member 20 may be laminated onto an interior or exterior perimeter region of the membrane 16 (creating a multi-layer structure), or the edge of the stiffing member 20 may be permanently joined to the edge of the membrane 16 (creating a single layer structure). In either configuration, the stiffening member 20 is fabricated from a material effective to provide a higher degree of rigidity, when compared with the stiffness of the membrane 16. In this way, the stiffening member 20 may be a comprised of one or more layers of material, either similar or dissimilar from the material forming the membrane 16. For example, if a sheet of 0.006″ polyvinyl chloride is chosen for the membrane 16, the stiffening member 20 may comprise an additional single perimeter layer of 0.006″ polyvinyl chloride, an additional single perimeter layer of 0.015″ phenyl ethane or a plurality of additional layers. In yet another embodiment, the stiffening member 20 may be comprised of a thin deformable metal layer or a semi rigid plastic material embedded with deformable metal components. Such a combination allows the stiffening member 20 to be generally flexible, yet permits a user to more accurately conform the cover 10 to the shape of the protected appendage (having “memory”). The stiffening member 20 serves to prevent the membrane 16 and seal 18 from bulging, cupping, twisting, or otherwise distorting from its desired general shape. This helps to ensure a constant interface is maintained between the seal 18 and the patient's skin.

Extending from the mounting tabs 12a and 12b are a plurality of mounting straps 22a and 22b. The mounting straps 22a and 22b may pass through slots 14a-14d, or they may be affixed to the cover 10 using mechanical fasteners, adhesive bonding, or chemical or thermal welding. In use, the straps 22a and 22b wrap around the patient's appendage, and cooperate to secure the cover 10 thereto. The straps 22a and 22b may be formed from an elastic or mesh material for enhanced comfort and breathability (perspiration permeability), but substantially solid or inelastic straps may be effective in certain configurations. Adjustment of attachment tension may be accomplished by fasteners known to the art. Buttons, snaps, buckles and through-holes, ratchets, or constricting lace systems may be used. However, using hook and loop type fasteners provides a high degree of adjustability at an economical price. In one embodiment, hooked fastener portions 24a and 24b form one end of straps 22a and 22b. Looped fastener portions 26a and 26b are affixed to the same side of a portion of the remaining length of straps 22a and 22b. In this embodiment, the looped fastener portions 26a and 26b utilize an elastic backing material for enhanced flexibility when worn by a patient. The elastic nature of the straps 22a and 22b is effective to maintain patient comfort while maintaining secure orientation of the cover 10, in the event of muscle flexure or appendage swelling.

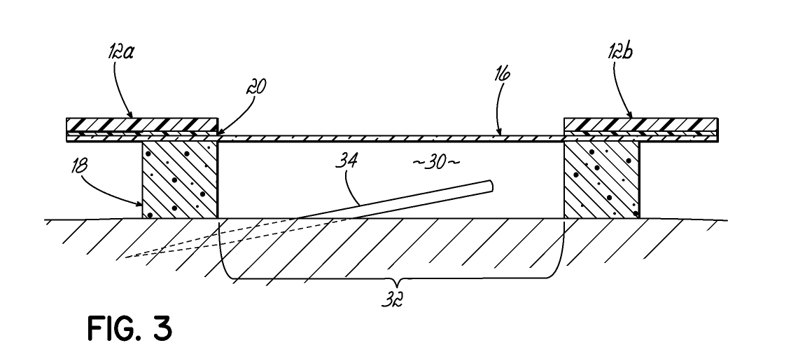

Referring to FIG. 3, a seal 18 with a square cross sectional area is laminated to the membrane 16. In this embodiment, the seal 18 and membrane 16 form a void 30 within the cover 10. More specifically, in this embodiment, the seal 18 is dimensioned such that the membrane 16 is supported in a non-contact relationship with the site 32 or an object 34 (shown here as an IV/catheter lure) penetrating the site. This configuration may be useful to prevent discomfort by preventing contact between the membrane 16 and the site (e.g. a burn or wound), or it may prevent trauma by isolating the object 34 from contact by exterior forces. Finally, the stiffening member 20 and mounting tabs 12a and 12b are shown as laminated to the seal 18 and membrane 16.

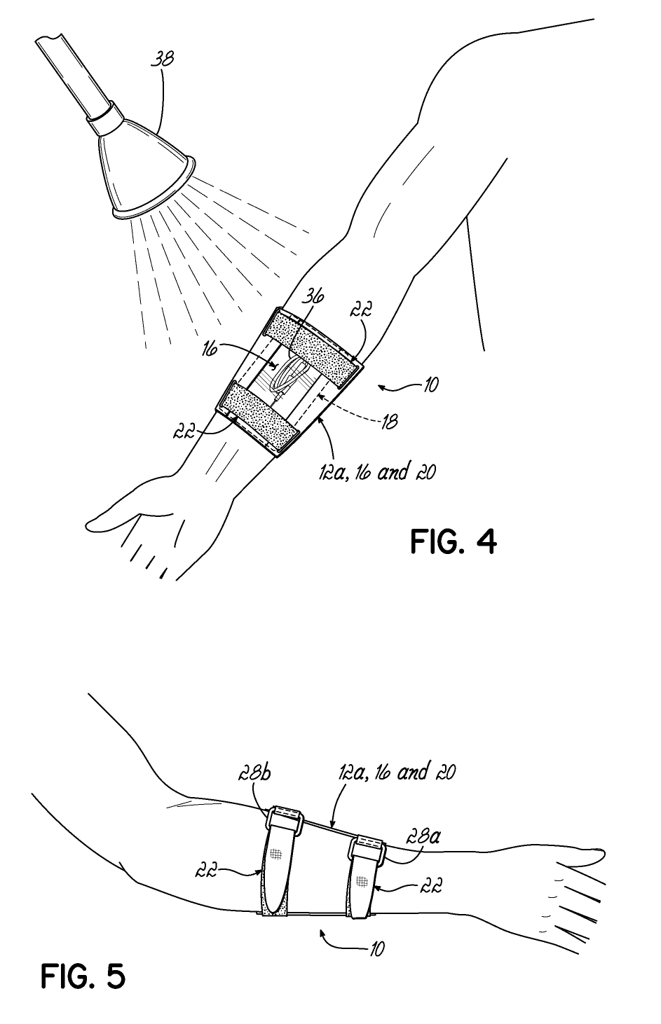

In use, and now referring to FIGS. 4-5, to secure the cover 10 on an appendage or region of the body, the straps 22a and 22b may be passed through fastening rings 28a and 28b disposed at the end opposite of the hooked portion 24a and 24b of the straps 22a and 22b. The fastening rings 28a and 28b allow straps 22a and 22b to be doubled back upon themselves, such that the hooked fastener portions 24a and 24b mate with the corresponding looped fastener portion 26a and 26b to secure the straps 22a and 22b around the patient. In one embodiment, the overall length of the straps 22a and 22b may be varied to accommodate the frustoconical taper inherent in appendage geometry. Generally, the shortest strap is located proximate to the hand, foot, etc. It should be noted that the method, numerosity, and configuration of attachment points may be varied to accommodate various applications.

In use, the cover may be positioned over the catheter/IV with all of the associated tubing completely confined within the interior volume formed by the membrane 16, seal 18, and skin of the patient (FIG. 4). Alternatively, a user may remove tubing from the luer of the catheter, cap the end, and utilize the cover 10 to protect the infusion site. As noted briefly above, the use of the cover 10 is not limited to protection of infusion sites. Wound sites and burn injuries are effectively protected from liquid or particulate contamination while the cover 10 is emplaced. Additionally, the seal 18 provides a resilient and impact absorbing perimeter around the wound or burn, and may prevent or reduce additional trauma if the site is physically impacted by another object. Likewise, with an appropriately dimensioned seal 18, surgical fixtures penetrating the skin, or trauma-related impalements, may also be protected from contamination by the cover 10.

IV tubing 36, stowed within the void 30 of the cover 10, is visible through the transparent window of the membrane 16. The membrane 16 and seal 18 (shown with hidden lines) protects the IV tubing from the contaminate source 38, while the straps 22a and 22b and stiffening member 20 facilitate consistent contact between the seal 18 (shown with hidden lines) and the site 32. Because of the adjustable nature of the straps 22a and 22b, the device is removable and suitable for reuse by the person to whom it is issued.

While the present invention has been illustrated by a description of various embodiments, and while these embodiments have been described in some detail, they are not intended to restrict or in any way limit the scope of the disclosed invention. Additional advantages and modifications will readily appear to those skilled in the art. The various features of the present invention may be used alone or in any combination depending on the needs and preferences of the user. This has been a description of the present invention, along with methods of practicing the present invention as currently known.

What is claimed is:

1. A patient site protective cover comprising:
a membrane;
a resilient and substantially impermeable seal around a perimeter of the membrane and dimensioned to surround a site of a patient; and
at least one strap operably coupled to the membrane and configured to secure the cover over the site.

2. The cover of claim 1, wherein the membrane is substantially impermeable to liquid, vapor, and particulate matter.

3. The cover of claim 1, wherein the membrane is differentially permeable and configured to allow vapor to pass through the membrane, and further configured to prevent liquids or particulate matter from passing through the membrane.

4. The cover of claim 1, wherein the membrane is configured to allow vapor and liquid to pass through the membrane, and further configured to prevent particulate matter from passing through the membrane.

5. The cover of claim 1, wherein the stiffening member is resilient and has memory, thereby allowing a user to substantially conform the cover to contours of the site.

6. The cover of claim 1 further comprising:
at least one buckle operably coupled to the at least one strap and configured to tension the at least one strap.

7. The cover of claim 1, wherein the at least one strap includes an elastic portion.

8. The cover of claim 1, wherein the at least one strap includes a hook and loop fastener system.

9. The cover of claim 1, wherein the seal is comprised of a plurality of protrusions or bulbs configured to improve the interface between the seal and the site.

10. The cover of claim 1, further including a stiffening member positioned around at least a portion of the perimeter of the membrane, the stiffening member configured to resist dimpling and distortion of the membrane and seal.

11. The cover of claim 1, wherein the seal is coated with a substance effective to improve the interface between the seal and the site.

12. The cover of claim 12 wherein the seal is dimensioned with a height effective to support the membrane in a non-contact relationship relative to the higher of one of the site, and an object penetrating the site.

13. A method of protecting a site on a patient from contamination, the method including the steps of:
surrounding the site with a substantially impermeable seal;
placing a membrane over the site and the seal;
bonding the seal, membrane, and stiffening member together; and
securing the seal, membrane, and stiffening member to the site with at least one strap.

14. The method of claim 13 wherein placing the membrane includes maintaining a non-contacting relationship between the membrane and the site.

15. The method of claim 13 wherein surrounding the site with a substantially impermeable seal includes dimensioning the seal to provide a void between the membrane and the higher of one of the site, and an object penetrating the site.

16. The method of claim 15 further including the step of storing a length of tubing, connected to an IV/catheter penetrating the site, within the void.

17. The method of claim 13 wherein placing the membrane includes selecting a membrane that is substantially impermeable to liquid, vapor, and particulate matter.

18. The method of claim 13 wherein placing the membrane includes selecting a differentially permeable membrane that is configured to allow vapor to pass through the membrane, and further configured to prevent liquids or particulate matter from passing through the membrane.

19. The method of claim 13 further including the step of utilizing a stiffening member positioned around at least a portion of a perimeter of the membrane, the stiffening member configured to resist dimpling and distortion of the membrane and seal.

20. A patient site protective cover comprising:
a membrane;
a resilient and substantially impermeable seal around a perimeter of the membrane and dimensioned to surround a site of a patient;
a stiffening member positioned around at least a portion of the perimeter of the membrane, the stiffening member configured to resist dimpling or distortion of the membrane;
a pair of mounting tabs, each mounting tab of the pair of mounting tabs positioned at a respective opposite side edge of the membrane, each mounting tab having at least one slot in each opposite end thereof; and
a pair of straps configured to secure the cover over the site, each strap of the pair of straps being threaded through the slots in respective ends of the mounting tabs.
